(19) United States
(12) Patent Application Publication
(10) Pub. No.: US 2003/0181615 A1
(43) Pub. Date: Sep. 25, 2003

(54) Bromosulphonated fluorinated cross-linkabke elastomers based on vinylidene fluoride having low t9 and processes for their preparation

(76) Inventors: Bruno Michel Ameduri, Montpellier (FR); Michel armand, Montreal (CA); Mario Boucher, St-Etienne-des-Gres (CA); Abdellatif Manseri, Montpellier (FR)

Correspondence Address: BURNS DOANE SWECKER & MATHIS L L P, POST OFFICE BOX 1404, ALEXANDRIA, VA, 22313-1404, US

(21) Appl. No.: 10/296,833

(22) PCT Filed: Jun. 12, 2001
(86) PCT No.: PCT/CA01/00878

(30) Foreign Application Priority Data
Jun. 13, 2000 (CA) 2,312,194

(51) Int. Cl. C08F 4/34; C07C 21/18; C08F 14/18

(52) U.S. Cl. 526/227; 526/232.1; 526/243; 526/247; 526/249; 526/253; 568/677; 570/135

(57) ABSTRACT

Compounds corresponding to formula (I)

F₂C═CFX(CY₂)ₙBr (I)

in which:

X represents an atom of oxygen or no atom;

Y represents an atom of hydrogen or of fluorine; and

n is a whole natural number ranging from 0 to 10 inclusive,

excluding bromotrifluoroethylene, 3-bromo-perfluoropropene, 4-bromo-1,1,2,-trifluorobutene, 4-bromo-perfluorobutene-1 and perfluoro(2-bromo-ethylvinyl ester), and their use in the synthesis of fluorinated copolymers then in the synthesis of homosulphonated fluorinated elastomers, exhibiting a low glass transition temperature.

### FIELD OF THE INVENTION

The present invention relates to bromosulphonated fluorinated cross-linkable elastomers based on vinylidene fluoride and possessing the special feature of exhibiting low glass transition temperatures (T_{g}). The present invention relates also to original processes in particular for the synthesis of cross-linkable elastomers with low glass transition temperatures (T_{g}) from copolymers, together with the use of such elastomers in the manufacture of stable parts intended in particular for the aeronautical, petroleum, automotive, mining and nuclear industries, and also plastics processing.

For example, such elastomers are used in the manufacture of stable parts such as membranes, polymeric electrolytes, ionomers, components of fuel cells supplied for example with hydrogen or methanol, gaskets, O-rings, radiator hoses, pipes, pump housings, diaphragms and piston heads.

Because of their chemical inertia, ion exchange membranes, either partially or totally fluorinated, are conventionally adopted in chlorine-soda processes or fuel cells consuming hydrogen or methanol. Such membranes are available commercially under names such as Nafion®, Flemion®, Dow®. Other similar membranes are proposed by Ballard Inc. in application WO 97/25369, which discloses, inter alia, copolymers of tetrafluoroethylene and perfluorovinyl ethers.

The term copolymer as used for the purpose of the present invention relates to compounds formed of macromolecules containing different monomeric patterns 2, 3, 4, 5, 6 or more in number. Such compounds with high molar masses are obtained when one or more monomers polymerise together. Examples of copolymers obtained in this way from 3, 4, 5 or 6 different monomer patterns [are] terpolymers, tetrapolymers, pentapolymers and hexapolymers, obtained respectively by terpolymerisation, tetrapolymerisation, pentapolymerisation and hexapolymerisation reactions.

### PRIOR ART

The known fluorinated elastomers exhibit a unique combination of properties (resistance to heat, to oxidation, to ultraviolet rays (UV), to ageing, to corrosive chemical agents, to motor fuels and to the absorption of water; low surface tensions, dielectric constants and refraction indices). The combination of these properties has enabled them to be used in “high tech” applications in numerous sectors: as gaskets (space industry, aeronautics), as semi-conductors (microelectronics), as radiator hoses, pipes, pump housings, piston heads, and diaphragms (chemical, automotive and petroleum industries).

Fluorinated elastomers (Progr. Polym. Sci. 26 (2001) 105-187, and in particular copolymers based on vinylidene fluoride (or 1,1-difluoroethylene, VDF) are polymers of choice for applications such as coatings or paints or more recently for membranes or components of fuel cells fed for example with hydrogen or methanol. These polymers are resistant to aggressive, reducing or oxidising conditions, and also to hydrocarbons, solvents, and lubricants (Progr. Polym. Sc. 14 (1989) 251 and 26 (2001) 105).

However, in order to improve their chemical inertia properties and their mechanical properties, it has appeared necessary to cross-link these elastomers. Elastomers based on VDF may be cross-linked by various methods (chemical in the presence of polyamines, polyalcohols and organic peroxides or ionising radiation or by electron bombardment), described in the reviews Progr. Polym. Sci. 26 (2001) 105, Rubber Chem. Technol. 55 (1982) 1004, in the work “Modem Fluoropolymers”, Chapter 32, p. 597, or in the article Angew. Makromol. Chem. 76/77 (1979) 39. It happens, however, that the products cross-linked by the polyamines or the polyalcohols do not possess the optimum characteristics for the applications in question [elastomers as gaskets or radiator hoses, diaphragms, pump housings for use in the automotive industry (Casaburo, Caoutchoucs et Plastiques, 753 (1996) 69)]. Cross-linking by peroxides has, based on fluoroiodised or fluorobrominated elastomers, yielded more encouraging results.

It should be noted that the fluorobrominated elastomers described in the literature are few in number. First of all, among the copolymers composed of fluorinated olefins and brominated alkenes, there are listed the pairs TFE/BrTFE (U.S. Pat. Nos. 4,035,565; 4,035,586 and 4,214,060 and the article J. Polym. Sc. Polym. Physics Ed., 23 (1985) 1099) and VDF/BrTFE (Polym. Bull. 11 (1984) 35). The terpolymers are more numerous, mainly compounds of VDF and hexa-fluoropropene (HFP) conferring on the terpolymers an elastomeric character and better thermal stability. The brominated monomers are: bromotrifluoroethylene (BrTFE) (U.S. Pat. Nos. 4,214,060 and 4,271,275); 1,1-difluoro-2-bromoethylene (WO 81/00573); 4-bromo-3,3,4,4-tetrafluorobutene (U.S. Pat. No. 4,214, 060) and trifluorovinylic ω-bromo ethers (Euro Pat. 0153848 and 0769521 and cited in the articles Kautsch. Gummi Kunst. 44 (1991) 833 and Rubber Chem. Technol. 55 (1982) 1004). The use of fluorinated olefins other than HFP has been mentioned in the patent U.S. Pat. No. 4,115,481. Finally, bromofluorinated tetrapolymers based on tetrafluoroethylene, HFP and VDF have been disclosed in the Canadian patent CA 2 182 328 (1997) and in the article Rubber Chem. Technol. 55 (1982) 1004.

However, various companies use trifluorovinyl ethers that are not brominated but functional and also containing ether bridges that favour a reduction in the glass transition temperature (T_{g}). These functional monomers have resulted in industrial products.

For example, the company DuPont markets Nafion membranes obtained by copolymerisation of TFE with the monomer F₂C═CFOCF₂CFCF₃)OC₂F₄SO₂F (PFSO₂). Similarly, the company Asahi Glass utilises this sulphonated monomer for the manufacture of Flemion® membrane. Other monomers of the same functionality, for example, F₂C═CFOCF₂CF(CF₃)OC₃F₆SO₂F (for the Aciplex® membrane, Asahi Chemical) F₂C═CFOC₂F₄SO₂F or of a carboxylate functionality such as the monomer F₂C═CFO[CF₂CF(CF₃)O]ₓC₂F₄CO₂CH₃ (for Nafion® and Aciplex® membranes when x equals 1, and for Flemion® membranes if x equals 0) are also used.

In addition, applications CA 2293846 and CA 2200622 disclose the easy copolymerisation of PFSO₂F with VDF, and applications CA 2293845 and CA 2 299 621 present PFSO₂F/VDF/HFP terpolymerisation. Moreover, the use of brominated monomers favours the cross-linking (by the peroxides) of the polymers formed and improves their thermal stability, their mechanical properties and their resistance to chemical agents, petroleum, strong acids and oxidation.

Recent studies relating to copolymerisation involve a fluorinated olefin (mainly TFE) and trifluorovinyl ethers.

It may be noted that most of the syntheses based on brominated monomers and trifluorovinyl ethers involve tetrafluoroethylene (TFE), e.g.TFE/perfluoromethyl vinyl ether/BrTFE, perfluoroallyl bromide, (U.S. Pat. Nos. 3,987,126 and 4,214,060) and PAVE/TFE/4-bromo-3,3,4,4-tetrafluorobutene (U.S. Pat. No. 4,973,634) terpolymers.

Two publications and five patents describe the terpolymerisation of fluorinated olefins with brominated or iodised monomers and PAVEs (mainly perfluoromethyl vinyl ether or perfluoro (2-bromo-ethyl vinyl ether). The international patent WO 9220743 (1992) proposes the synthesis of VDF/HFP/F₂O═CFO(CF₂)ₙ terpolymers (where n=0 to 5 inclusive) obtained in the presence of transfer agent 1,4-diiodoperfluorobutane, then are cross-linked with peroxides. In addition, Canadian patent 2,068,754 (1992) deals with HFP/VDF/TFE/PMVE/ethylene pentapolymers the T_{g} values of which vary from −9 to −18° C. and up to −28° C. when the monomer F₂C═CFOC₂F₄Br also participates in this polymerisation. Similarly, cross-linkable elastomers based on HFP, VDF, TFE and the aforementioned brominated monomer have been disclosed in European patent EP 410351 (1991), Canadian patent 2182328 (1997) and in the articles by Apotheker et coll., Kautsch. Gummi Kunst. 44 (1991) 833. In addition, European patent EP 0079555 describes VDF/trifluorovinyl ethers/perfluoro (2-bromo ethyl vinyl ether) terpolymers.).

### SUMMARY OF THE INVENTION

The present invention describes the preparation of novel bromofluorinated monomers, then the copolymerisation of trifluorovinyl monomers with brominated terminations with fluorinated monomers. This process leads to the synthesis of novel copolymers, then of novel cross-linkable sulphonated bromofluorinated elastomers exhibiting very low glass transition temperatures (T_{g}), good resistance to acids, petroleum and motor fuels and good properties of use.

### DETAILED DESCRIPTION OF THE INVENTION

The first subject of the present invention comprises the family of compounds corresponding to formula I:

F₂C═CFX (CY₂)ₙBr (I)

in which:

X represents an atom of oxygen or no atom;

Y represents an atom of hydrogen or fluorine, and

n is a whole natural number varying between 0 and 10 inclusive.

According to a preferred embodiment, the present invention comprises the sub-family of compounds corresponding to formula II:

F₂C═CF(CH₂)ₙBr (II)

in which:

n is a whole natural number varying between 0 and 10 inclusive.

The second subject of the present invention comprises a process for preparing a fluorinated copolymer by radical copolymerisation, said process comprising the reaction:

of a compound corresponding to formula I:

F₂C═CFX(CY₂)ₙBr (I)

in which:

X represents an atom of oxygen or no atom;

Y represents an atom of hydrogen or fluorine, and

n is a whole natural number varying between 0 and 10 inclusive,

with a compound corresponding to formula III₁:

F₂C═CFOR_{F1} (III₁)

in which R_{F1} denotes:

a linear or branched group of formula CₙF₂ₙ₊₁ (n denoting a whole natural number ranging from 1 to 10) or

with a compound corresponding to formula III₂:

F₂C═CFOR_{F2}—G (III₂)

in which R_{F2} denotes:

a linear or branched group of formula CₙF₂ₙ (n denoting a whole natural number ranging from 1 to 10), and

in which G represents:

a functional group SO₂F, CO₂R (R is the group CₚH₂ₚ₊₁, in which p is a whole natural number varying between 0 and 5) or a functional group P(O)(OR′) in which R′ is independently an atom of hydrogen or an alkyl group as C₁-C₅.

A preferred embodiment of the process according to the present invention comprises a process for preparing a fluorinated copolymer, by reaction:

of a compound corresponding to formula II′:

F₂C═CFBr (II′)

with the compound of formula III₁ or III₂ as defined previously,

so as to obtain a statistical copolymer corresponding to formula IV:

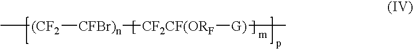

in which:

R_{F} represents the groups R_{F1} and R_{F2} defined previously, the group G being absent when R_{F} represents R_{F1}, and

in which:

n, m and p represent independently whole natural numbers such that the ratio n/m ranges from 1 to 25 and such that p ranges from 10 to 300, preferably, the ratio n/m varies from 2 to 23 and p ranges from 15 to 200, more preferably still, the ratio n/m ranges from 6 to 19 and p ranges from 20 to 100.

Another preferred embodiment of the invention comprises a process for preparing fluorinated copolymer, by reaction:

of a compound corresponding to formula II″:

F₂C═CF(CH₂)₂Br (II″)

with a compound of structure III₁ or III₂ as defined previously,

so as to obtain a statistical copolymer corresponding to formula V:

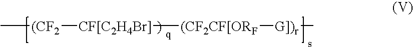

in which:

R_{F} represents the groups R_{F1} and R_{F2} defined previously, the group G being absent when R_{F} represents R_{F1}, and

in which:

q, r and s represent independently whole natural numbers such that the ratio q/r ranges from 1 to 20 and s ranges from 10 to 300, preferably, the ratio q/r ranges from 2 to 15 and s ranges from 15 to 200, more preferably still, the ratio q/r ranges from 2 to 10 and s ranges from 20 to 100.

Another subject of the present invention comprises a copolymerisation process, comprising the reaction:

of a compound corresponding to formula II′:

F₂C═CFBr (II′)

with a compound corresponding to formula III₁:

F₂C═CFOR_{F1} (III₁)

in which R_{F1} denotes:

a linear or branched group of formula CₙF₂ₙ₊₁ (n being a natural number ranging from 1 to 10) or

with a compound corresponding to formula III₂:

F₂C═CFOR_{F2}—G (III₂)

in which R_{F2} denotes:

a linear or branched group of formula CₙF₂ₙ (n being a natural number ranging from 1 to 1 0), and

in which G represents:

a functional group SO₂F, CO₂R with R being the group CₚH₂ₚ₊₁, in which p represents a whole natural number ranging from 0 to 5) or being a functional group P(O)(OR′) in which R′ denotes independently an atom of hydrogen or a C₁-C₅ alkyl group, and

with:

a compound corresponding to formula VI:

FCX═CYZ (VI)

in which X, Y and Z:

represent independently atoms of hydrogen, fluorine, chlorine or groups of formula CₙF₂ₙ₊₁ (with n equalling 1, 2 or 3), but X, Y and Z cannot represent an atom of fluorine simultaneously,

so as to obtain a statistical copolymer corresponding to formula VII:

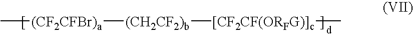

in which:

R_{F} represents the groups R_{F1} or R_{F2} defined previously, the group G being absent when R_{F} represents R_{F1}, and

in which:

a, b, c and d represent independently whole natural numbers such that the ratio b/a ranges from 0.1 to 15, such that the ratio b/c ranges from 1 to 20 and such that d ranges from 10 to 200, according to a preferred embodiment, the ratio b/a ranges from 1 to 10, the ratio b/c ranges from 1 to 15 and d ranges from 15 to 150. According to an even more advantageous embodiment, the ratio b/a ranges from 2 to 6, the ratio b/c ranges from 2 to 9 and d ranges from 25 to 100.

A preferred embodiment of the copolymerisation process comprises the reaction:

of a compound corresponding to formula II″:

F₂C═CF(CH₂)₂Br (II″)

with a compound corresponding to formula III₁:

F₂C═CFOR_{F1} (III₁)

in which R_{F1} denotes:

a linear or branched group of formula CₙF₂ₙ₊₁ (n being a whole natural number ranging from 1 to 10) or

with a compound corresponding to formula III₂:

F₂C═CFOR_{F2}—G (III₂)

in which R_{F2} denotes:

a linear or branched group of formula CₙF₂ₙ (n being a whole natural number ranging from 1 to 10), and

in which G represents:

a functional group SO₂F, CO₂R with R being the group CₚH₂ₚ₊₁, in which p represents a whole natural number ranging from 0 to 5 or being a functional group P(O)(OR′) in which R′ independently is an atom of hydrogen or an alkyl group as C₁-C₅ and

with:

a compound corresponding to formula VI:

FCX═CYZ (VI)

in which:

X, Y and Z independently represent atoms of hydrogen, fluorine, chlorine or groups of formula CₙF₂ₙ₊₁ (with n equalling 1, 2 or 3), but X, Y and Z cannot represent a fluorine atom simultaneously,

so as to obtain a statistical copolymer corresponding to formula VIII:

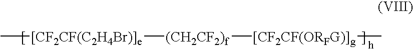

in which:

R₄ represents the groups R_{F1} and R_{F2} defined previously, the group G being absent when R_{F} represents R_{F1}, and

in which:

e, f, g and h represent independently whole natural numbers such that the ratio f/e ranges from 1 to 10, such that the ratio f/g ranges from 1 to 10 and such that h ranges from 10 to 250, the ratio f/g advantageously ranges from 2 to 8 and h ranges from 15 to 200, according to a still more preferred embodiment the ratio f/e ranges from 1 to 3, the ratio f/g ranges from 3 to 7 and h ranges from 20 to 150.

This copolymerisation process is preferably carried out in (“batch type”) tanks and the reaction is conducted in an emulsion, microemulsion, suspension or solution.

According to another preferred embodiment of the invention, the reaction is initiated in the presence of at least one organic radical initiator chosen preferably from the group composed of peroxides, peresters, percarbonates, alkyl peroxypivalates and diazoic compounds.

According to another preferred embodiment of the copolymerisation process according to the invention, the reaction is carried out in the presence of:

at least one peroxide chosen preferably from the group composed of t-butyl peroxide, t-butyl hydroperoxide and t-butyl peroxypivalate and t-amyl peroxypivalate, and/or

at least one perester which is preferably benzoyl peroxide, and/or

at least one percarbonate, which is preferably t-butyl cyclohexyl peroxydicarbonate.

According to a particularly advantageous embodiment of the present invention for the carrying out of the copolymerisation reaction, the concentration of peroxide and/or of perester and/or of percarbonate in the reaction medium is such that the initial molar ratio between the initiator and the monomers ([initiator]ₒ/[monomers]ₒ) lies between 0.1 and 2%, and preferably between 0.5 and 1%, the initiator being the compound with the formula tBuO—OtBu or tBuO—OC(O)tBu and the monomers being the compounds of formula I, II, III₁, III₂, II′, II″ and VI, the expression [initiator]ₒ expresses the initial molar concentration of initiator and the expression [monomers]ₒ expresses the total initial concentration of monomers.

The copolymerisation reaction is conducted preferably:

in the presence of t-butyl peroxypivalate and at a reaction temperature which lies between 70 and 80° C., preferably at a temperature of about 75° C., or

in the presence of t-butyl peroxide and at a reaction temperature of between 135 and 145° C., preferably at a temperature of about 140° C.

The copolymerisation process is preferably carried out in solution in the presence of at least one organic solvent, which is advantageously chosen from the group composed of perfluoro-n-hexane, acetonitrile or mixtures of perfluoro-n-hexane and acetonitrile.

The amount of solvent in the reaction medium is preferably such that the initial ratio by weight between the solvent and the monomers lies between 0.5 and 1.5, more preferably still between 0.6 and 1.2.

According to another advantageous embodiment of the copolymerisation process according to the invention, the reaction is conducted with an initial molar ratio between the initiator and the monomers ([initiator]ₒ/[monomers]ₒ) of between 0.1 and 2%, and preferably between 0.5 and 1%. The initiator being the compound with the formula tBuO—OtBu or tBuO—OC(O)tBu and the monomers being the compounds of formula I, II, III₁, III₂, II′, II″ and VI as defined previously. The expression [initiator]ₒ expresses the initial molar concentration of initiator and the expression [monomers]ₒ expresses the total initial concentration of monomers.

According to another preferred embodiment of the invention the copolymerisation process is implemented with a reagent with formula III₁ or III₂, which is preferably perfluoro(4-methyl-3,6-dioxaoct-7-ene) sulphonyl fluoride (PFSO₂F) and with the compound of formula VI that is preferably vinylidene fluoride (VDF).

A third object of the present invention comprises fluorinated polymers, preferably fluorinated copolymers capable of being obtained by one of the processes defined in the preceding part relating to the second subject of the present invention.

A fourth object of the present invention comprises bromofunctional fluorinated copolymers capable of being obtained by any one of the processes defined in the preceding part relating to the second subject of the present invention.

According to an advantageous embodiment of the invention, the bromofunctional fluorinated copolymers contain from 7 to 24% of bromotrifluoroethylene (BrTFE); from 20 to 30% of perfluoro(4-methyl-3,6-diaxaoct-7-ene) sulphonyl fluoride (PFSO₂F); and from 56 to 73% of vinylidene fluoride (VDF).

According to another embodiment of the invention, the bromofunctional fluorinated copolymers contain from 2 to 15% of 1,1,2-trifluoro-4-bromobutene (BrEF); from 20 to 30% of perfluoro(4-methyl-3,6-diaxaoct-7-ene) sulphonyl fluoride (PFSO₂F); and from 65 to 77% of vinylidene fluoride (VDF).

Among the bromofunctional fluorinated copolymers previously defined, those which possess the following chemical functions or fluorinated groups:

—SO₂F;

—OCF₂CF(CF₃)OCF₂CF₂SO₂F;

—tBuO—CF2—CH₂—;

—CH₂CF₂—CH₂CF₂—CH₂—CF₂—;

—CH₂CF₂—CH₂CF₂—CF₂—CH₂—;

tBuO—CH₂CF₂—CH₂CF₂—;

—CH₂CF₂—(CF₂—CH₂—CF₂—;

—CF₂CF(OR_{F}SO₂F)—CH₂CF₂—CF₂C_{F}(OR_{F}SO₂F)—;

—CH₂CF₂—CF₂CF(OR_{F}SO₂F)—;

—OCF₂CF(CF₃)OCF₂CF₂SO₂F;

—CH₂CF₂—CH₂CF₂—CF₂CH₂—;

—CH₂CF₂—CF₂CH₂—CH₂CF₂—;

—CF₂CFBrCH₂CF₂;

—CH₂CF₂—CF₂CF(OR_{F}SO₂F)—CH₂CF₂—;

—CH₂CF₂—CF₂CF(OR_{F}SO₂F)—CH₂CF₂—;

—(CF₂CFBr)ₙ—;

—CH₂CF₂—CF₂CF(OR_{F}SO₂F)—CF₂CH₂—; and

—OCF₂CF(CF₃)OC₂F₄SO₂F;

associated respectively with the following chemical shifts, expressed in ppm, in NMR of ¹⁹F:

+45;

−77 to −80;

−83;

−91;

−95;

−102;

−103 to 105;

−108;

−110;

−112;

−113;

−116;

−118;

−122;

−125;

−126;

−127; and

−144,

are of particular interest.

Among the bromofunctional fluorinated copolymers previously defined, those which possess the following chemical functions or fluorinated groups:

—SO₂F;

—OCF₂CF(CF₃)OCF₂CF₂SO₂F;

tBuO—CF₂—CH₂—;

—CH₂CF₂—CH₂CF₂—CH₂CF₂—;

—CF₂CF(RF)—CH₂CF₂—CH₂CF₂—;

—CF₂CF (R_{F})—CH₂ CF₂(CH₂CF₂—CF₂CF₂—;

—CH₂CF₂—CH₂CF₂—CF₂CH₂—;

—CF₂CF(OR_{F}SO₂F)—CH₂CF₂—CF₂CF(OR_{F}SO₂F)—;

—CH₂CF₂—CH₂CF₂—CF₂CF(R₄)—;

—OCF₂CF(CF₃)OCF₂CF₂SO₂F;

—CH₂CF₂—CH₂CF₂—CF₂CH₂—;

—CH₂CF₂—CF₂CH₂—CH₂CF₂—;

—CH₂ CF₂—CF₂CF(C₂H₄Br)—CH₂CF₂—;

—CF₂CF(OR_{F}—SO₂F)—CF₂CF(C_{2H}₄Br)—CH₂—CF₂—;

—CH₂CF₂—CF₂CF(OR_{F}SO₂F)—CH₂CF₂—;

—CH₂CF₂—CF₂CF(OR_{F}SO₂F)—CH₂CF₂—;

—CH₂CF₂—CF₂CF(OR_{F}SO₂F)—CF₂CH₂—;

—OCF₂CF(CF₃)OC₂F₄SO₂F;

—CH₂CF₂—CF₂CF(C₂H₄Br)—CH₂CF₂—;

—CH₂CF₂—CF₂CF(C₂H₄Br)—CF₂—;

associated respectively with the following chemical shifts, expressed in ppm, in NMR of ¹⁹F:

+45;

−77 to −80;

−83;

−91;

−92;

−93;

−95;

−108;

−110;

−112;

−113;

−116;

−119;

−120;

−122;

−125;

−127;

−144;

−161 to −165 and

−178 to −182

are also of particular interest.

A fifth subject of the present application comprises a process for preparing a bromosulphonated fluorinated elastomer, characterised in that any one of the polymers obtained according to one of the processes of the invention is subjected to a cross-linking stage carried out preferably in the presence of at least one peroxide (preferably in a concentration of between 1 and 5%) and/or in the presence of at least one triallylisocyanurate (preferably in a concentration of between 5 and 20%) followed by a hot post-cross-linking stage, i.e. at 200-220° C.

A sixth subject of the present application comprises the bromosulphonated fluorinated elastomers capable of being obtained by the processes, which constitute the fifth subject of the present invention.

A preferred family of the elastomeric compounds according to the invention comprises bromosulphonated fluorinated elastomers having very low glass transition temperatures (T_{g}), these glass transition temperatures, which are measured according to the standard ASTM E-1356-98, are preferably between −45 and −18° C., more preferably still, between −35 and −21° C.

The bromosulphonated fluorinated elastomers are, in addition, characterised in that they exhibit:

an inherent viscosity measured according to the standard ASTM D-2857-95 that is preferably between 0.8 and 1.8 mL/g; and/or

a thermal stability (ATG) of preferably up to 325° C. in air, at which temperature 5% weight loss is measured.

A seventh subject of the present application comprises the use of one or more of the cross-linkable bromosulphonated fluorinated elastomers according to the invention, for:

the manufacture of membranes, polymeric electrolytes, ionomers, components of fuel cells supplied, for example, with hydrogen or methanol;

the making of gaskets and O-rings, radiator hoses, pipes, pump housings, diaphragms, piston heads (finding applications in the aeronautical, petroleum, automotive, mining, nuclear industries) and

for plastics processing (aid processing products).

An eighth subject of the present invention comprises a process for cross-linking the sulphonyl groups of a sulphonated polymer chosen from the family of the bromosulphonated fluorinated elastomers defined in the fifth and sixth subjects of the present invention. During the implementation of this process, at least one fraction of the cross-linking bonds carries an ionic charge. The process also includes the bringing of said sulphonated polymer into contact with a cross-linking agent permitting the reaction between two sulphonyl groups originating from adjacent polymer chains, in order to form said cross-linking bonds.

In general, the invention describes the synthesis of original fluorinated elastomeric copolymers based on either commercial fluorobrominated monomers (such as bromotrifluoroethylene BrTFE) or synthetic fluorobrominated monomers (such as 1,1,2-trifluoro-4-bromobutene, BrEF) and containing functional perfluoroalkyl vinyl ether and optionally other fluorinated alkenes. The originality of this invention comes in particular from the following characteristics:

1) The preparation of co-brominated trifluorovinylic monomers, reagents of co-polymerisation with commercial fluorinated alkenes or functional fluorinated monomers;

2) The synthesis of fluorinated elastomers based on functional perfluoroalkyl vinyl ethers and/or functional perfluoroalkoxy vinyl ethers and optionally other fluorinated alkenes, is carried out with VDF instead of tetrafluoroethylene, the latter being widely used for the manufacture of fluorinated elastomers;

3) The synthesis of the fluorinated elastomers in said invention requires the use of monomers bearing siloxane groups, the latter contributing in general to a reduction in the glass transition temperature (T_{g});

4) The cross-linkable fluorinated elastomers obtained by the present invention comprise a minority of fluorobrominated monomers of the structure XYC═CZW(CT₂)ₓBr (with X, Y, Z able to be chosen from among atoms of hydrogen, or of halogens with at least one atom of fluorine, W representing an atom of oxygen or no atom; T symbolising an atom of hydrogen or of fluorine and x being a natural number of between 0 and 10 inclusive) and a majority of functional perfluoroalkyl vinyl ether (PAVE) or functional perfluoro-alkoxyalkyl vinyl ether (PAAVE) for the copolymers; and a minority of fluorobrominated monomers and a majority of VDF or of functional perfluoroalkyl vinyl ether or functional perfluoroalkoxyalkyl vinyl ether according to the initial molar ratios of these two fluorinated monomers for the terpolymers;

5) The fluorinated elastomers synthesised by said invention exhibit very low glass transition temperatures (T_{g}), said elastomers thus being able to be used in the field of plastics processing (as “aid processing products”), or other leading-edge industries (aerospace, electronics or automotive industries, petroleum, transport of corrosive, acid or very cold fluids such as liquid nitrogen, oxygen and hydrogen). In addition, joints with high thermal resistance may be prepared from these elastomers;

6) These bromosulphonated fluorinated elastomers are easily cross-linkable with peroxides. This cross-linking significantly improves their properties of resistance to oxidation, solvents, hydrocarbons, motor fuels, acids and aggressive media.

Furthermore, it is well known that perfluorinated polymers may not conventionally be cross-linked using techniques traditionally employed for non-fluorinated polymers because of the easy elimination of the fluoride ion and the steric hindrance of the perfluorinated chains. However, the general technique described in the application PCT WO99/38897, the contents of which are incorporated by reference, makes it possible to create cross-linkings, i.e. bonds, between the sulphonyl groups attached to the adjacent polymer chains, including those having a perfluorinated skeleton, for example those derived from the following monomer and its copolymers:

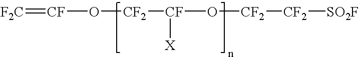

in which

X is F, Cl or CF₃;

n is 0 to 10 inclusive.

Advantageously, cross-linking may be carried out while the polymer is in the form of a non-ionic polymer precursor, but after having been moulded or pressed into the desired form. This consequently results in a mechanically much stronger material. The present invention also relates to the moulding or pressing of the cross-linked polymer in the form of membranes or hollow fibres (hereinafter “membranes”) for use in a fuel cell, electrolyser in water, a chlorine-soda process, electrosynthesis, water treatment or ozone production. The use of the cross-linked polymers as catalysts for certain chemical reactions, owing to the strong dissociation of the ionic groups introduced by the cross-linking technique and the insolubility of the polymer chain, also forms part of the invention.

The creation of stable cross-linkings is brought about by the intervention of a reaction between two —SO₂L groups originating from adjacent polymer chains. The reaction is initiated by a cross-linking agent, and permits the formation of derivatives according to the following formulae:

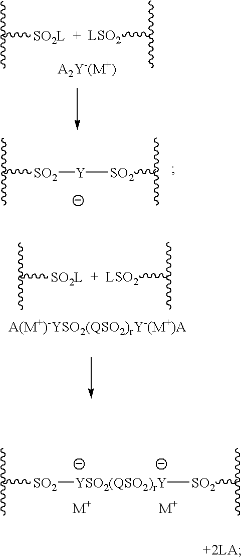

in which:

r is 0 or 1;

M comprises an inorganic or organic cation;

Y comprises N or CR in which R comprises H, CN, F, SO₂R³, C₁₋₂₀ alkyl substituted or non-substituted; C₁₋₂₀ substituted or non-substituted; C₁₋₂₀ alkylene substituted or non-substituted, in which the substituent comprises one or more atoms of hydrogen, and in which the chain comprises one or more substituents F, SO₂R, aza, oxa, thia or dioxathia;

R³ comprises F, C₁₋₂₀ alkyl substituted or non-substituted; C₁₋₂₀ aryl substituted or non-substituted; C₁₋₂₀ alkylene substituted or non-substituted, in which the substituent comprises one or more halogen atoms;

Q comprises a divalent radical C₁₋₂₀ alkyl, C₁₋₂₀ oxaalky, C₁₋₂₀ azaalkyl, C₁₋₂₀ thiaalkyl, C₁₋₂₀ aryl or C₁₋₂₀ alkylaryl, each being able to be optionally substituted by one or more halogen atoms, and in which the chain comprises one or more substituents oxa, aza or thia;

A comprises M, Si(R′)₃, Ge(R′)₃ or Sn(R′)₃ in which R′ is C₁₋₁₈ alkyl;

L comprises an unstable group such as a halogen atom (F, Cl, Br), an electrophilic heterocycle N-imidazolyl, N-triazolyl, R²SO₃ in which R² is an optionally halogenated, organic radical, and

R² comprises the proton; the alkyl, alkenyl, oxaalkyl, oxaalkenyl, azaalkyl, azaalkenyl, thiaalkyl, thiaalkenyl, dialkylazo, optionally hydrolysable silaalkyl, optionally hydrolysable silaalkenyl radicals, said radicals being able to be linear, branched or cyclic and containing from 1 to 18 carbon atoms; the aliphatic cyclic or heterocyclic radicals with 4 to 26 carbon atoms containing optionally at least one side chain containing one or more heteroatoms such as nitrogen, oxygen or sulfur; the aryls, arylalkyls, alkylaryls and alkenylaryls with 5 to 26 carbon atoms including optionally one or more heteroatoms in the aromatic ring or in a substituent.

The cross-linking reaction may involve all of the sulphonyl groups or only a fraction of the latter. The cross-linking reagents may be added or used according to various techniques well known to the average person skilled in the art. Advantageously, the polymer is moulded in the desired form prior to the cross-linking, for example in the form of membranes or hollow fibres, and the material is immersed or covered with a solution of the cross-linking agent in one or more solvents promoting the coupling reaction.

If only a fraction of the bonds forming the bridge between the polymer chains is required, the remaining —SO₂L groups may be hydrolysed in the conventional manner in the form of sulphonate by alkaline hydrolysis.

The cross-linked polymer obtained using the process of the present invention may be easily separated from the secondary products of the reaction, which are for example volatile, such as (CH₃)₃SiF or (CH₃)₃SiCl. Alternatively, the cross-linked polymer may be washed with the aid of an appropriate solvent such as water or an organic solvent in which it is insoluble. In addition, traditional techniques well known to the average person skilled in the art, for example ion exchange or electrophoresis, may be used to exchange the cation M⁺ obtained in the cross-linking reaction and/or coming from the non-cross-linking ionogenic agent for the cation desired for the final application.

### ADVANTAGE OF THE INVENTION COMPARED WITH THE PRIOR ART

The advantages associated with the present invention are mainly the following:

1. Use of commercial brominated olefins and/or preparation of original fluorobrominated monomers by simple synthetic means;

2. Fluorobrominated monomers which are copolymerisation reagents are used;

3. The synthesis process is carried out in batch mode;

4. The process in said invention is carried out in solution and uses traditional readily commercially available organic solvents;

5. The process of said invention comprises radical polymerisation in the presence of traditional readily commercially available initiators;

6. Tetrafluoroethylene (TFE) is not used in this invention;

7. The perfluorinated olefin that forms part of the composition of the fluorinated elastomers prepared by said invention is vinylidene fluoride (VDF); the latter is far less expensive and much less dangerous than TFE and provides on the elastomers obtained good resistance to oxidation, chemical agents, polar solvents and petroleum and a reduction in the glass transition temperature;

8. The fluorinated elastomers in said invention may be prepared from the monomer PFSO₂F whose copolymerisation with BrTFE (or BrEF) and VDF has never been the subject of work described in the literature. In addition, this monomer sulphonated by means of its sulphonyl fluoride function makes the creation of cross-linking sites in these elastomers possible;

9. The fluorinated copolymers obtained by this process exhibit very low glass transition temperatures varying between −35 to −21° C.;

10. These bromosulphonated fluorinated copolymers may be easily cross-linked by means of peroxides, thus resulting in materials that are stable, inert and insoluble in all solvents, hydrocarbons or strong acids.

The present invention also relates to the synthesis of reactive ω-brominated trifluorovinylic monomers and the obtaining of bromofluorinated elastomers based on VDF and PAVE, then the study of their cross-linking, and their field of application. The cross-linking of these fluorobrominated copolymers is carried out in the presence of peroxide and triallylisocyanurate whose general mechanism is discussed in the article Rubber Chem. Technol. 55 (1982) 1004 and the review Prog. Polym. Sci. 26 (2001) 105-187. However, to our knowledge no study relating to the copolymerisation of PFSO₂F with brominated alkenes and other fluorinated olefins has been described in the literature.

Synthesis of ω-brominated Trifluorovinyl Monomers

The first objective of this invention comprises the making available of novel trifluorovinyl monomers that are reagents of copolymerisation with fluorinated olefins and which exhibit a brominated termination. This objective is achieved by compounds corresponding to formula I:

F₂C═CFX(CY)ₙBr (I)

in which:

X represents an atom of oxygen or no atom;

Y represents an atom of hydrogen or fluorine;

n is a whole natural number of between 0 and 10 inclusive.

More particularly, the present invention proposes compounds corresponding to formula II:

F₂C═CF(CH₂)ₙBr (II)

in which:

n is as defined above.

Preparation of the Bromosulphonated Fluorinated Elastomers

Within the scope of the present invention, all the types of processes generally used, such as microemulsion, bulk, suspension and solution polymerisation may be used.

Solution polymerisation is preferably used, however.

The various fluorinated alkenes employed possess at most four atoms of carbon and have the structure R₁R₂C═CR₃R₄ where the Rᵢ, i groups, i being a whole number from 1 to 4 inclusive, are such that at least one of the Rᵢ ‘s is fluorinated or perfluorinated. This therefore encompasses: vinyl fluoride (VF), vinylidene fluoride (VDF), trifluoroethylene, chlorotrifluoroethylene (CTFE), 1-hydropentafluoro-propylene, hexafluoroisobutylene, 3,3,3,-trifluoropropene and in general all fluorinated or perfluorinated vinyl compounds. In addition, perfluorovinyl ethers also play the role of comonomers. Among them, there may be mentioned the perfluoroalkyl vinyl ethers (PAVE) whose alkyl group has between one and three carbon atoms, for example, perfluoromethyl vinyl ether (PMVE), perfluoroethyl vinyl ether (PEVE) and perfluoropropyl vinyl ether (PPVE). These monomers may also be perfluoroalkoxy alkyl ethers (PAAVE), disclosed in U.S. Pat. No. 3,291,843 and in the journals Prog. Polym. Sci., M. Yamabe et coll. vol. 12 (1986) 229 and B. Améduri et coll., vol. 26 (2001) 105, such as perfluoro(2-n-propoxy)-propyl vinyl ether, perfluoro-(2-methoxy)-propyl vinyl ether; perfluoro(3-methoxy)-propyl vinyl ether, perfluoro-(2-methoxy)-ethyl vinyl ether, perfluoro-(3,6,9-trioxa-5,8-dimethyl)-dodeca-1-ene, perfluoro-(5-methyl-3,6-dioxo)-1-nonene. In addition, perfluoroalkoxyalkyl vinyl ether monomers with carboxylic terminations or with a sulphonyl fluoride termination, such as perfluoro(4-methyl-3,6-dioxaoct-7-ene) sulphonyl fluoride, may also be used for the synthesis of fluorinated elastomers that is described in this invention. Mixtures of PAVE and PAAVE may be present in the copolymers.

More particularly, perfluoro(4-methyl-3,6-dioxaoct-7-ene) sulphonyl fluoride (PFSO₂F) has been used as comonomer.

The brominated monomers employed in this invention are olefins in which at least one of the hydrogen atoms has been replaced by a bromine atom and, optionally, one or more remaining atoms of hydrogen have been replaced by an atom of another halogen, mainly fluorine. Some of these monomers are commercial ones, such as vinyl bromide, bromotrifluoroethylene (BrTFE), 1-bromo-2,2-difluoroethylene, 4-bromo-3,3,4,4-tetrafluoro-1-butene, 3-bromo-3,3-difluoro-propane, 1,1,2-trifluoro-3-bromo-1,3-butadiene or 2-bromoperfluoroethyl-perfluorovinyl ether. Other fluorobrominated olefins such as 3-bromopentafluoro-1-propene, 1,1,2-trifluoro-3-methyl-4-bromopentene, 4-bromo-3,4-dichloro-3,4-difluoro-1-butene, 6-bromo-5,5,6,6-tetrafluoro-1-hexene, 4-bromo-3-trifluoromethyl-1-butene, 1-bromo-1,1-difluoro-2-butene may be prepared by methods such as those described by Tarrant and Gillman (J. Am. Chem. Soc. 76 (1954) 3466 and 5423), by Tarrant and Tandon (J. Org. Chem. 34 (1969) 864), by Fainberg and Miller (J. Am. Chem. Soc. 79 (1957) 4170) or by Hu and coll. (J. Fluorine Chem. 66 (1994) 171). However, we have also synthesised 4-bromo-1,1,2-trifluoro-1-butene, which, to our knowledge, has not been the subject of papers.

The solvents employed to carry out the polymerisation in solution are as follows:

esters of formula R—COO—R′ where R and R′ are hydrogenated or alkyl groups able to contain between 1 and 5 carbon atoms, but also hydroxy (OH) groups or OR″ ether groups where R″ is an alkyl containing from 1 to 5 carbon atoms. More particularly, R═H or CH₃ and R′=CH₃, C₂H₅, i-C₃H₇, t-C₄H₉;

fluorinated solvents of the type: ClCF₂CFCl₂, C₆F₁₄, n-C₄F₁₀₃ perfluoro-2-butyltetrahydrofurane (FC 75), and

acetone, 1,2-dichloroethane, isopropanol, tertiobutanol, acetonitrile or butyronitrile.

The solvents preferably employed are methyl acetate and acetonitrile in variable quantities.

The reaction temperature range may be determined by the decomposition temperature of the initiator and varies from 20 to 200° C. The temperatures preferably employed lie between 55 and 80° C.

In the process according to the invention, polymerisation may be initiated by the intervention of the conventional initiators of radical polymerisation. Representative examples of such initiators are azoic initiators (such as azobisisobutyronitrile, AIBN), dialkyl peroxydicarbonates, acetylcyclohexane sulphonyl peroxide, aryl or alkyl peroxide such as dibenzoyl peroxide, dicumyl peroxide, t-butyl peroxide, t-alkyl perbenzoates and t-alkyl peroxypivalates. Preference is nevertheless given to dialkyl peroxides (preferably t-butyl peroxide), to dialkyl peroxy-dicarbonates, such as diethyl and diisopropyl peroxydicarbonates, and to t-alkyl peroxypivalates such as t-butyl and t-amyl peroxypivalates and, more particularly, to t-alkyl peroxypivalates.

For the emulsion polymerisation process, we employed a wide range of cosolvents used in various proportions in the mixture with water. Similarly, various surfactants were used.

One of the polymerisation processes used may also be microemulsion, as described in European patent EP 250767, or dispersion, as indicated in U.S. Pat. No. 4,789,717 or the European patents 196904; 280312 and 360292. The content of these documents is incorporated by reference to the present application.

The reaction pressures vary between 2 and 120 bar according to the test conditions.

Chain transfer agents may be generally used to regulate and principally reduce the molar masses of the copolymers. Among the latter, mention may be made of telogens containing from 1 to 10 carbon atoms and possessing terminal bromine or iodine atoms such as, for example, compounds of the type R_{F}X (where R_{F} is a perfluorinated group with the formula CₙF_{2b+1}, n=1 to 10 inclusive, X denoting a bromine or iodine atom) or XR_{F}′X (with R_{F′}=(CF₂)ₙ where n=1 to 6 inclusive) or alcohols, ethers, esters. A list of the various transfer agents used in the telomerisation of fluorinated monomers is given in the journal “Telomerization reactions of fluoro-alkanes”, B. Améduri and B. Boutevin in the work “Topics in current chemistry” (Ed. R. D. Chambers, vol. 192 (1997), p. 165, Springer Verlag 1997.

The whole range of relative percentages of the various copolymers that can be synthesised from the fluorinated monomers employed and leading to the formation of the fluorinated copolymers has been studied (Tables 1 and 2).

The products were analysed by NMR of ¹H and ¹⁹F in deuterated acetone or DMF. This method of analysis allowed the percentages of the comonomers introduced into the products to be unambiguously determined. For example, we established perfectly, from the micro-structures characterised in the literature (Polymer 28 (1987) 224; J. Fluorine Chem. 78 (1996) 145; and applications CA 2293846, CA 2299 622, CA 2293 845, CA 2299621), the relations between the characteristic signals of the copolymers BrTFE/PFSO₂F (see Table 3) and the copolymers BrTFE (or BrEF)/PFSO₂F/VDF (Tables 3 and 4) as NMR of ¹⁹F and the structure of the products. This analysis highlights BrTFE/PFSO₂F, VDF/PFSO₂F and BrTFE/VDF (or BrEF/VDF) diads, and the head-to-tail and head-to-head linkages of the blocks of VDF units (respectively at −91 and −113, −116 ppm).

The molar percentages of the various monomers in the VDF/PFSO₂F/BrTFE copolymers were determined by means of equations 1, 2 and 3 given below (table 3).

$$\begin{matrix}molar \% of VDF = \frac{A}{A + B + C} & Equation 1 \\ molar \% of BrTFE = \frac{B}{A + B + C} & Equation 2 \\ molar \% of {PFSO}_{2} F = \frac{C}{A + B + C} & Equation 3\end{matrix}$$

in which:

A=I₋₈₃+I₋₉₁+I₋₉₅+I₋₁₀₂+I₋₁₀₈+I₋₁₁₀+I₋₁₁₃+I₋₁₁₆+I₋₁₂₇

B=2(I₋₁₁₈+I₋₁₂₆)

C=I₋₁₂₂

where I₋ᵢ is the value of the integration of the signal situated at −i ppm on the NMR spectrum of ¹⁹F.

The molar percentages of the various monomers in the copolymers VDF/PFSO₂F/BrEF were determined from equations 4, 5 and 6 given below (table 4).

$$\begin{matrix}molar \% of VDF = \frac{D}{D + E + F} & Equation 4 \\ molar \% of BrEF = \frac{E}{D + E + F} & Equation 5 \\ molar \% of {PFSO}_{2} F = \frac{F}{D + E + F} & Equation 6\end{matrix}$$

in which:

D=I₋₉₁+I₋₉₂+I₋₉₃+I₋₉₅+I₋₁₀₈+I₋₁₁₀+I₋₁₁₃+I₋₁₁₆+I₋₁₂₇

E=I₋₁₁₉+I₁₂₀)

F=I₁₂₂

where I₋ᵢ is the value of the integration of the signal situated at −i ppm on the NMR spectrum of ¹⁹F.

By differential calorimetric analysis (DSC), we note that the copolymers BrTFE/PFSO₂F with high content of BrTFE (more than 85%) are crystalline, unlike the copolymers BrTFE (or BrEF)/PFSO₂F/VDF, which exhibit only one glass transition temperature (T_{g}) and an absence of fusion temperature (Tables 3 and 4). These low values of T_{g} testify to an increased elastomeric character, particularly original in the case of bromofluorinated polymers.

At the same time, the thermal stabilities (ATG), obtained in air, of these bromosulphonated fluorinated copolymers are highly satisfactory.

Cross-linking of the Bromosulphonated Fluorinated Elastomers

The elastomers of this invention may be cross-linked by using systems based on peroxides and triallylisocyanurate when such copolymers contain atoms of iodine and/or of bromine in terminal position of the macromolecule. Peroxidic systems are well known, such as those described in European patent EP Appl. 136 596 or in the reviews Kaut. Gummi Kunst. 44 (1991) 833, Rubber World 207 (1993) 18 and Rubber Chem. Technol. 55 (1982) 1004. The vulcanisation of these elastomers may also be carried out by ionic, radiation or electron bombardment methods such as those described in U.S. Pat. Nos. 3,876 654 and 4,259,463, European patent 335705 or the journal Prog. Polym. Sci. 26 (2001) 105 or those cited above.

The copolymers of such compositions may find applications in the preparation of O-rings, pump housings, diaphragms possessing a very good resistance to motor fuels, gasoline, t-butyl methyl ether, alcohols, engine oils and strong acids (HCl, HNO₃ and H₂SO₄), combined with good elastomeric properties, in particular a very good resistance at low temperatures. These copolymers also exhibit the advantage of being cross-linkable in the presence of traditionally used agents.

### EXAMPLES

The following examples are given in order to better illustrate the present invention, but they can under no circumstances constitute a limitation to the scope of said invention.

### Example 1

Synthesis of 1,2-dibromo-2-chlorotrifluoroethane

A Carius tube (internal diameter: 78 mm, thickness: 2.5 mm and length 220 mm) containing a magnetic bar, 175 g (1.1 mol.) of bromine and 1.1 g (0.006 mol.) of benzophenone is cooled in a liquid nitrogen/acetone mixture (−80° C.). After having performed three blank/nitrogen cycles, 131 g (1.12 mol.) of chlorotrifluoroethylene (CTFE) are introduced. The reaction commences with the addition of the CTFE. The tube is sealed, then progressively heated to −40° C., the exothermicity of the reaction being controlled by cooling the tube in the bath to −80° C. After discoloration of the reaction crude, the solution is agitated at ambient temperature under UV for one hour. Distillation leads to 175 g of colourless liquid (T_{Eb}=90-92° C.) with a yield of 91%.

¹⁹F NMR (CDCl₃) δ: −60.1 (system AB, ²J_{FF}=166.8 Hz, ³J_{FF}=13.5 Hz, ³J_{FF}=15.0 Hz, BrCF₂, 2F); −69.4 (part X of a system ABX, ³J_{FF}=13.1 Hz, ³J_{FF}=14.7 Hz, CFCl, 1F).

### Example 2

Ethylenation of 1,2-dibromo-2-chlorotrifluoroethane

The following are introduced into the 1 litre Hastelloy reactor equipped with mechanical agitation means (hollow Hastelloy blades, i.e. turbine with gaseous effect), a manometer, two valves (gas inlet and salting out), and a rupture disc, and situated in a thermocontrolled jacket, 465.5 g (1.68 mol.) of BrCF₂CFClBr, 6.5 g (0.016 mol.) of bis (4-tertiobutylcyclohexyl)-carboxydicarbonate and 200 g of tertiobutanol. The reactor is sealed, degassed then vacuumed and cooled to −80° C. in an acetone/liquid nitrogen mixture. 66 g (2.35 mol.) of ethylene are introduced. After this the reactor is allowed to return to ambient temperature, then progressively heated to 60° C., which generates a violent exothermal reaction reaching 115° C. at the end of 25 minutes and leading to a pressure maximum of 28 bar. This pressure falls progressively, corresponding to the consumption of ethylene. The reaction is left thus at 60° C. for 2 hours. After cooling to ambient temperature, the reactor is cooled in the ice, then progressively degassed. The solvent is evaporated and the CPV chromatogram of the crude shows the total conversion of 1,2-dibromo-1,1,2-trifluoro-2-chloroethane. The overall yield is 85%. After distillation, first of all the colourless mono-ethylenated derivative, 1,4-dibromo-2-chloro-1,1,2-trifluorobutane (BrCF₂CFClC₂H₄Br), (T_{Eb}=59-62° C./20 mm Hg), then the colourless diethylenated derivative, 1,6-dibromo-2-chloro-1,1,2-trifluorohexane (BrCF₂CFClC₄H₈Br) (T_{Eb}=50-53° C./0.8 mm Hg), is recovered.

NMR Characterisation of the Monoethylenated Derivative

¹H NMR (CDCl₃: δ=2.8 (q, CFClCH₂CH₂Br, 2H); 3.5 (t, ³J_{HH}=6.9 Hz, −6.9 Hz, —CH₂Br, 2H).

¹⁹F NMR (CDCl₃): δ=−61.5 (system AB, BrCF₂—, 2F); −118.5 (part X of a system/ABX, CFCl—, 1F).

NMR Characterisation of the Diethylenated Derivative

¹H NMR (CDCl₃): δ=1.9 (m, CFClCH₂C₂H₄CH₂Br, 4H); 2.4 (q, CFClCH₂C₃H₆Br, 2H); 3.4 (t, CFClC₃H₆CH₂Br, 2H).

The NMR spectrum of the ¹⁹F of the diethylenated derivative is identical to that of the monoethylenated one.

### Example 3

Synthesis of 1,1,2-trifluoro-4-bromobutene (F₂C═CFC₂H₄Br)

A solution consisting of 90.3 g (0.297 mol.) of 1,4-dibromo-2-chloro-1,1,2-trifluorobutane in 40 g of DMSO was added drop by drop, at 40° C., to a twin-necked flask fitted with a cooling apparatus and containing an agitated solution composed of 21.34 g (0.326 mol.) of zinc, 6.62 g (0.048 mol.) of ZnCl₂ and 130 g of DMSO. After the addition, the reaction-agitated crude was heated to 90° C. and kept at this temperature for 4 hours. After cooling, the crude was treated with an acid solution (HCl 10%) then neutralised with NaHCO₃ and washed with water. Extraction with ClCF₂CFCl₂ (F-113), followed by drying on MgSO₄, resulted, after distillation of the F-113, in 20.2 g of F₂C═CFC₂H₄Br (1,1,2-trifluoro-4-bromobutene), corresponding to a yield of 36%. T_{Eb}=92-95° C. (colourless liquid).

¹H NMR (CDCl₃): δ system AA′BB′; 2.82 (ddt, CH₂Br, 2H); 3.48 (t, ⁹J_{HH}=6.9 Hz, CH₂CH₂Br).

¹⁹F NMR (CDCl₃) δ: −103.5 (ddt, ²J_{FaFb}=82.8 Hz, ³J_{FaFc}=33.3 Hz; ⁴J_{FaH}=2.5 Hz; Fₐ); −123.0 (ddq, ²J_{FbFa}=82.8 Hz, ³J_{FbFc}=114.3 Hz, ⁴J_{FbH}=3.7 Hz; F_{b}); −177.6 (ddt, ³J_{FcFb}=114.2 Hz, ³J_{FcFa}=33.1 Hz, ³J_{FcH}=21.0 Hz; F_{c}).

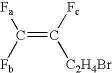

### Examples 4 to 7

Radical Copolymerisation of BrTFE with PFSO₂F and Radical Copolymerisation VDF/BrTFE/F₂O═CFOCF₂CF(CF₃)OC₂F₄SO₂F

25.0 g (0.056 mol.) of F₂C═CFOCF₂CF(CF₃)OC₂F₄SO₂F, 0.61 g (0.003 mol.) of tertiobutyl peroxypivalate and 55.0 g of acetonitrile were introduced into a 160 ml Hastelloy reactor, fitted with two valves, with a safety disc and a manometer (Example 5, Table 1). The reactor is sealed, vacuumed and cooled in an acetone/liquid nitrogen mixture. Once the temperature reaches −80° C., 8.8 g (0.048 mol.) of bromotrifluoroethylene then 16.3 g (0.25 mol.) of vinylidene fluoride is introduced in succession. The reactor is allowed to return to ambient temperature, then heated to 75° C. in an oil bath for 15 hours. After cooling to ambient temperature then in ice, the reactor is degassed. The acetonitrile is partially evaporated, then the copolymer is precipitated by slow drop-wise addition into 200 ml of strongly agitated cold pentane. The copolymer sticks to the walls of the erlenmeyer and after decanting, separation and drying in a vacuum at 80° C. to constant weight, 31 g of orange-coloured, highly viscous product is obtained. The chemical shifts of the fluorinated groups of the copolymers (Table 3) were determined unambiguously from all the polymers obtained, the test details and the results for which are given in table 1.

Differential calorimetric analysis (DSC), using a Perkin Elmer Pyris 1 apparatus calibrated with indium and with octadecane, on an approx. 15 mg sample, was carried out using the following cycle three times: heating from −100° C. to +165° C. (at 40 then 20° C./min)/cooling from +165° C. to −100° C. (at 20° C./min). The results for the copolymers revealed a single glass transition temperature (T_{g}) corresponding to the point of inflection of the enthalpic leap. The second and third cycles yielded reproducible values for T_{g}.

The thermogravimetric analyses (TGA) were carried out by means of a TGA 51-133 apparatus made by Texas Instruments, in air, with a heating rate of 10° C./min.

### Examples 8 to 10

Synthesis of Bromosulphonated Fluorinated Elastomers by Radical Copolymerisation VDF/F₂C═CFC₂H₄Br/F₂C═CFOCF₂CF(CF₃)OC₂F₄SO₂F

In the case of Example 10 (Table 2), we used a 160 ml Hastelloy reactor (identical to that used above) into which 5.4 g (0.028 mol.) of F₂C═CFC₂H₄Br, 31.0 g (0.069 mol.) of F₂C═CFOCF₂CF(CF₃)OC₂F₄SO₂F, 0.22 g (0.0015 mol.) of tertiobutyl peroxide and 30.0 g of acetonitrile were introduced. The reactor was sealed, then vacuumed and cooled in an acetone/liquid nitrogen mixture. Once the temperature reached −80° C., 14.0 g (0.218 mol.) of vinylidene fluoride (VDF) were introduced. The reactor was allowed to return to ambient temperature, then heated to 135° C. for 18 hours. After cooling in ice, the reactor was degassed and 3.2 g of VDF, which had not reacted was salted out (the rate of conversion of VDF was 77%). The characterisation by NMR of the ¹⁹F of the reaction crude shows that 82% of the sulphonated monomer reacted (the presence of the characteristic signal centred at −138.5 ppm indicates the presence of the sulphonated monomer which has not totally reacted).The acetonitrile was partially evaporated, then. as in the previous example; the copolymer was precipitated by drop-wise addition into 200 ml of strongly agitated cold pentane. After decanting, separation and drying in a vacuum at 80° C. to constant weight, 38 g of orange-coloured, highly viscous product was obtained. The yield by weight was 75%. The NMR spectrum of the ¹⁹F made it possible to recognise unambiguously the molar percentages of the three comonomers from the characteristic signals of the various fluorinated groups contained in the patterns composed of VDF (66.3%), PFSO₂F (28.9%) and brominated monomer BrEF (4.8%) (Table 4). Differential calorimetric analysis (DCA) showed the absence of a peak, attributed to fusion, but the presence of an enthalpic leap attributed to a single glass transition temperature (T_{g}=−35° C.). A thermogravimetric analysis carried out under air at 10° C./min has shown that this copolymer looses about 5% of its weight at 275° C. The test details and the results for the other examples are summarised in Table 2. The NMR analyses of the ¹⁹F characterising different chemical shifts of the various groups are given in table 4.

### Example 11

Cross-linking of the Bromosulphonated Fluorinated Copolymers, Based on BrTFE

2.00 g of the copolymer described in Examples 4 to 7 are dissolved in 20.0 g of acetone (Normapure). 0.050 g (0.0004 mol.) of 2,5-dimethyl-2,5-bis-(t-butylperoxy) of hexane and 0.12 g (0.0004 mol.) of triallylisocyanurate (or 2,4,6-triallyloxy-1,3,5-triazine) are added. When the solution is homogeneous, the acetone is evaporated, then the viscous residue is spread out in a mould situated between two sheets of PTFE, pressed (2 bar) at 175° C. for 20 min, then at 200° C. for 2 hours. The film obtained is clear, homogeneous and insoluble in all organic solvents and hydrocarbons and in concentrated HCl and H₂SO₄.

### Example 12

Cross-linking of the Bromosulphonated Fluorinated Copolymers Based on 4-bromo-1,1,2-trifluorobutene

According to the same procedure as in Example 11, 2.00 g of copolymer described in Examples 8 to 10 are dissolved in 20.1 g of acetone. 0.06 g (0.0004 mol.) of 2,5-dimethyl-2,5-bis(tert-butylperoxy)hexane and 0.12 g of triallylisocyanurate are mixed with the latter. When the solution is homogeneous, the acetone is evaporated and the viscous residue is poured into a mould, then hot-pressed (according to Example 11) between two sheets of PTFE. The film obtained is clear, homogeneous and insoluble in all solvents, hydrocarbons and strong acids.

TABLE 1
Operating conditions and results of the radical copolymerisations using BrTFE

| | | | | | | | | | | | | Conversion | | | | |
|---|---|---|---|---|---|---|---|---|---|---|---|---|---|---|---|---|
| | Mass | Mass | Mass | Mass | | Initial | Initial | Initial | VDF | PFSO₂F | BrTFE | rate | | Yield by | | T_{deg} 5% |
| Exam- | VDF | PFSO₂F | BrTFE | solvent | Cₒ | VDF | PFSO₂F | BrTFE | copo. | copo. | copo. | PFSO₂F | gas | wt | T_{g} | air |
| ple^{a} | g | g | g | g | % | mol. % | % | % | % | % | % | % | % | % | ° C. | ° C. |
| 4 | 0 | 3.2 | 6.8 | 9.2 | 0.7 | 0 | 14.4 | 85.6 | 0 | 4.0 | 96.0 | n.c. | | n.c. | n.c. | n.c. |
| | | | | C₄F₁₄ | P.P. | | | | | | | | | | | |
| 5 | 16.3 | 25.0 | 8.8 | 55.0 | 1.0 | 70.5 | 16.0 | 13.5 | 65.5 | 15.7 | 18.8 | 50 | 52 | 40.0 | −20.5 | 310 |
| | | | | CH₂CN | P.P. | | | | | | | | | | | |
| 6 | 16.5 | 25.0 | 5.0 | 55.1 | 0.8 | 75.5 | 16.5 | 8.0 | 66.6 | 22.0 | 11.4 | 57 | 75 | 65.0 | −27.6 | 280 |
| | | | | CH₃CN | P.P. | | | | | | | | | | | |
| 7 | 12.0 | 39.5 | 4.0 | 55.0 | 0.8 | 63.0 | 29.6 | 7.4 | 64.5 | 22.0 | 13.5 | 45 | 81 | 80.0 | −23.1 | 320 |
| | | | | CH₃CN | P.P. | | | | | | | | | | | |

^{a}Temperature of 75° C., period of 15 hours
P.P. = t-butyl peroxypivalate
n.c, = not calculated
Cₒ = [initiator]ₒ/([VDF)ₒ + [PFSO₂F]ₒ + [BrTFE]ₒ)

TABLE 2
Operating conditions and results of the radical copolymerisations using BrEF

| | Mass | Mass | Mass | Mass | | Initial | Initial | Initial | VDF | PFSO₂F | BrEF | Conversion rate | | | | T_{deg} 5% |
|---|---|---|---|---|---|---|---|---|---|---|---|---|---|---|---|---|
| | VDF | PFSO₂F | BrEF | solvent | Cₒ | VDF | PFSO₂F | BrEF | copo. | copo. | copo. | PFSO₂F | gas | Yield by wt | T_{g} | air |
| Example^{a} | g | g | g | g | % | mol. % | % | % | % | % | % | % | % | % | ° C. | ° C. |
| 8 | 20.0 | 33.0 | 2.3 | 30.0 | 0.5 | 78.5 | 18.4 | 3.0 | 78.0 | 20.0 | 2.0 | 64 | | 33 | −31 | 325 |
| | | | | CH₃CN | P.P. | | | | | | | | | | | |
| 9 | 18.0 | 30.0 | 2.0 | 30.1 | 0.5 | 78.5 | 18.7 | 2.8 | 74.5 | 23.4 | 2.1 | 78 | 73 | 65 | −35 | 295 |
| | | | | CH₃CN | tBu | | | | | | | | | | | |
| 10 | 14.0 | 31.0 | 5.4 | 30.0 | 0.6 | 69.0 | 22.0 | 8.8 | 66.6 | 28.9 | 4.8 | 82 | 77 | 75 | −35 | 275 |
| | | | | CH₃CN | tBu. | | | | | | | | | | | |

^{a}Temperature of 75° C. with the t-butyl peroxypivalate and 135° C. with the t-butyl peroxide, period of 15 hours
P.P. = t-butyl peroxypivalate
tBu = t-butyl peroxide
Cₒ = [initiator]ₒ/([VDF)ₒ + [PFSO₂F]ₒ + [BrEF]ₒ)

TABLE 3
NMR characterisation of the ¹⁹F of
the VDF/PFSO₂F/BrTFE copolymers

| | Chemical shift |
|---|---|
| Structure | (ppm) |
| —SO₂F | +45 |
| —OCF₂CF(CF₃)OCF₂CF₂SO₂F | −77 to −80 |
| tBuO—CF2—CH₂— | −83 |
| —CH₂CF₂—CH₂CF₂—CH₂CF₂— | −91 |
| —CH₂CF₂—CH₂CF₂—CF₂—CH₂— | −95 |
| tBuO—CH₂CF₂—CH₂CF₂ | −102 |
| —CH₂CF₂—(CF₂CFBr)ₙ— | −103 to 105 |
| —CF₂CF(OR_{F}SO₂F)—CH₂CF₂—CF₂CF(OR_{F}SO₂F)— | −108 |
| —CH₂CF₂—CF₂CF(OR_{F}SO₂F)— | −110 |
| —OCF₂CF(CF₃)OCF₂CF₂SO₂F | −112 |
| —CH₂CF₂—CH₂CF₂—CF₂CH₂— | −113 |
| —CH₂CF₂—CF₂CH₂—CH₂CF₂— | −116 |
| —CF₂CFBrCH₂CF₂ | −118 |
| —CH₂CF₂—CF₂CF(OR_{F}SO₂F)—CH₂CF₂— | −122 |
| —CH₂CF₂—CF₂CF(OR_{F}SO₂F)—CH₂CF₂— | −125 |
| —(CF₂CFBr)ₙ— | −126 |
| —CH₂CF₂—CF₂CF(OR_{F}SO₂F)—CF₂CH₂— | −127 |
| —OCF₂CF(CF₃)OC₂F₄SO₂F | −144 |

TABLE 4
NMR characterisation of the ¹⁹F
of the VDF/PFSO₂F/BrEF copolymer

| | Chemical |
|---|---|
| Structure | shift (ppm) |
| —SO₂F | +45 |
| —OCF₂CF(CF₃)OCF₂CF₂SO₂F | −77 to −80 |
| tBuO—CF2—CH₂— | −83 |
| —CH₂CF₂—CH₂CF₂—CH₂CF₂— | −91 |
| —CF₂CF(R_{F}) —CH₂CF₂—CH₂—CF₂— | −92 |
| —CF₂CF(R_{F})—CH₂CF₂—CH₂CF₂—CF₂CF )R_{F})— | −93 |
| —CH₂CF₂—CH₂CF₂—CF₂CH₂— | −95 |
| —CF₂CF(OR_{F}SO₂F)—CH₂CF₂—CF₂CF(OR_{F}SO₂F)— | −108 |
| —CH₂CF₂—CH₂CF₂—CF₂CF(R_{F})— | −110 |
| —OCF₂CF(CF₃)OCF₂CF₂SO₂F | −112 |
| —CH₂CF₂—CH₂CF₂—CF₂CH₂— | −113 |
| —CH₂CF₂—CF₂CH₂—CH₂CF₂— | −116 |
| —CH₂CF₂—CF₂CF(C₂H₄Br)—CH₂CF₂— | −119 |
| —CF₂CF(OR_{F}—SO₂F—CF₂CF(C₂H₄Br)—CH₂CF₂— | −120 |
| —CH₂CF₂—CF₂CF(OR_{F}SO₂F)—CH₂CF₂— | −122 |
| —CH₂CF₂—CF(OR_{F}SO₂F)—CH₂CF₂— | −125 |
| —CH₂CF₂—CF₂CF(OR_{F}SO₂F)—CF₂CH₂— | −127 |
| —OCF₂CF(CF₃)OC₂F₄SO₂F | −144 |
| —CH₂CF₂—CF₂CF(C₂H₄Br)—CH₂CF₂— | −161 to −165 |
| —CH₂CF₂—CF₂CF(C₂H₄Br)—CF₂— | −178 to −182 |

1. Compound corresponding to formula I:
F₂C═CFX(CY₂)ₙBr (I)
in which:
X represents an atom of oxygen or no atom;
Y represents an atom of hydrogen or of fluorine; and
n is a whole natural number varying between 0 and 10 inclusive,
excluding bromotrifluoroethylene, 3-bromo-perfluoropropene, 4-bromo-1,1,2,-trifluorobutene, 4-bromo-perfluorobutene-1 and perfluoro(2-bromo-ethylvinyl ester).

2. Compound according to claim 1, corresponding to formula II:
F₂C═CF(CH₂)ₙBr (II)
in which:
n is a natural number varying between 0 and 10 inclusive.

3. Process for preparing a fluorinated copolymer by radical copolymerisation, said process comprising the reaction of a compound corresponding to formula I:
F₂C═CFX(CY₂)ₙBr (I)
in which:
X represents an atom of oxygen or no atom;
Y represents an atom of hydrogen or of fluorine; and
n is a whole natural number varying between 0 and 10 inclusive,
with a compound corresponding to formula III₁:
F₂C═CFOR_{F1} (III₁)
in which R_{F1} is:
a linear or branched group of formula CₙF₂ₙ₊₁ (n denoting a whole natural number ranging from 1 to 10); or
with a compound corresponding to formula III₂:
F₂C═CFOR_{F2}—G (III₂)
in which R_{F2} is:
a linear or branched group of formula CₙF₂ₙ (n denoting a whole natural number ranging from 1 to 10); and
in which G represents:
a functional group SO₂F, CO₂R (R is the group CₚH₂ₚ₊₁, in which p is a whole natural number ranging from 0 to 5) or a functional group P(O)(OR′) in which R′ is independently an atom of hydrogen or a C₁-C₅ alkyl group.

4. Process for preparing a fluorinated copolymer according to claim 3, by reaction:
of a compound corresponding to formula II′:
F₂C═CFBr (II′)
with the compound of formula III₁ or III₂ as defined in claim 3,
so as to obtain a statistical copolymer corresponding to formula IV: 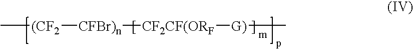
in which:
R_{F} represents the groups R_{F1} and R_{F2} defined in claim 3, the group G being absent when R_{F} represents R_{F1}, and
in which:
n, m and p represent independently whole natural numbers such that the ratio n/m ranges from 1 to 25 and such that p ranges from 10 to 300, preferably the ratio n/m ranges from 2 to 23 and p ranges from 15 to 200, more preferably still the ratio n/m ranges from 6 to 19 and p ranges from 20 to 100.

5. Process for preparing a fluorinated copolymer according to claim 3, by reaction of a compound corresponding to formula II″:
F₂C═CF(CH₂)₂Br (II″)
with a compound of structure III₁ or III₂ as defined in claim 3 so as to obtain a statistical copolymer corresponding to formula V: 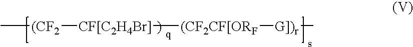
in which:
R_{F} represents the groups R_{F1} and R_{F2} defined in claim 3, the group G being absent when R_{F} represents R_{F1}; and
in which:
q, r and s represent independently whole natural numbers such that the ratio q/r ranges from 1 to 20 and s ranges from 10 to 300, preferably the ratio q/r ranges from 2 to 15 and s ranges from 15 to 200, more preferably still the ratio q/r ranges from 2 to 10 and s ranges from 20 to 100.

6. Copolymerisation process, comprising the reaction:
of a compound corresponding to formula II′:
F₂C═CFBr (II′)
with a compound corresponding to formula III₁:
F₂C═CFOR_{F1} (III₁)
in which R_{F1} is:
a linear or branched group of formula CₙF₂ₙ₊₁ (n being a whole natural number ranging from 1 to 10); or
with a compound corresponding to formula III₂:
F₂C═CFOR_{F2}—G (III₂)
in which R_{F2} is:
a linear or branched group of formula CₙF₂ₙ (with n being a natural number ranging from 1 to 10); and
in which G represents:
a functional group SO₂F, CO₂R with R being the group CₚH₂ₚ₊₁, in which p represents a whole natural number ranging from 0 to 5, or being a functional group P(O)(OR′) in which R′ is independently an atom of hydrogen or a C₁-C₅ alkyl group; and
with:
a compound corresponding to formula VI:
FCX═CYZ (VI)
in which:
X, Y and Z represent independently atoms of hydrogen, fluorine, chlorine or groups of formula CₙF₂ₙ₊₁ (n equalling 1, 2 or 3), but X, Y and Z cannot simultaneously represent an atom of fluorine,
so as to obtain a statistical copolymer corresponding to formula VII: 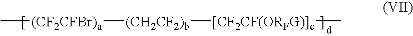
in which:
R_{F} represents the groups R_{F1} or R_{F2} defined previously in claim 3, the group G being absent when R_{F} represents R_{F1}; and
in which:
a, b, c and d represent independently whole natural numbers such that the ratio b/a ranges from 0.1 to 15, such that the ratio b/c ranges from 1 to 20 and such that d ranges from 10 to 200, preferably the ratio b/a ranges from 1 to 10, the ratio b/c ranges from 1 to 15 and d ranges from 15 to 150, and more preferably still the ratio b/a ranges from 2 to 6, the ratio b/c ranges from 2 to 9 and d ranges from 25 to 100.

7. Copolymerisation process comprising the reaction:
of a compound corresponding to formula II″:
F₂C═CF(CH₂)₂Br (II″)
with a compound corresponding to formula III₁:
F₂C═CFOR_{F1} (III₁)
in which R_{F1} denotes:
a linear or branched group of formula CₙF₂ₙ₊₁ (with n being a whole natural number ranging from 1 to 10); or
with a compound corresponding to formula III₂:
F₂C═CFOR_{F2}—G (III₂)
in which R_{F2} denotes:
a linear or branched group of formula CₙF₂ₙ (n being a whole natural number ranging from 1 to 10); and
in which G represents:
a functional group SO₂F, CO₂R with R being the group CₚH₂ₚ₊₁, in which p represents a whole natural number ranging from 0 to 5 or being a functional group P(O)(OR′) in which R′ is independently an atom of hydrogen or a C₁-C₅ alkyl group; and
with a compound corresponding to formula VI:
FCX═CYZ (VI)
in which:
X, Y and Z represent independently atoms of hydrogen, fluorine, chlorine or groups of formula CₙF₂ₙ₊₁ (n equalling 1, 2 or 3), but X, Y and Z cannot simultaneously represent a fluorine atom,
so as to obtain a statistical copolymer corresponding to formula VIII: 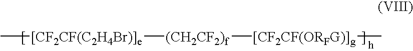
in which:
R₄ represents the groups R_{F1} and R_{F2} defined previously in claim 3, the group G being absent when R_{F} represents R_{F1}; and
in which:
e, f, g and h independently represent whole natural numbers such that the ratio f/e ranges from 1 to 10, such that the ratio f/g ranges from 1 to 10 and such that h ranges from 10 to 250, preferably the ratio f/e ranges from 1 to 5, the ratio f/g ranges from 2 to 8 and h ranges from 15 to 200, more preferably still the ratio f/e ranges from 1 to 3, the ratio f/g ranges from 3 to 7 and h ranges from 20 to 150.

8. Copolymerisation process according to claim 6 or 7, characterised in that the reaction is carried out in batch.

9. Copolymerisation process according to any one of claims 6 to 8, characterised in that the reaction is conducted in emulsion, microemulsion, suspension or solution.

10. Copolymerisation process according to any one of claims 6 to 9, characterised in that the reaction is initiated in the presence of at least one organic radical initiator chosen preferably from the group constituted by peroxides, peresters, percarbonates, alkyl peroxypivalates and diazoic compounds.

11. Copolymerisation process according to any one of claims 6 to 10, characterised in that the reaction is carried out in the presence of:
at least one peroxide chosen preferably from the group constituted of t-butyl peroxide, t-butyl hydroperoxide and t-butyl peroxypivalate and t-amyl peroxypivalate, and/or
at least one perester which is preferably benzoyl peroxide, and/or
at least one percarbonate, which is preferably t-butyl cyclohexyl peroxydicarbonate.

12. Copolymerisation process according to claim 11, characterised in that the concentration of peroxide and/or of perester and/or of percarbonate in the reaction medium is such that the initial molar ratio between the initiator and the monomers ([initiator]ₒ/[monomers]ₒ) lies between 0.1 and 2%, and preferably between 0.5 and 1%, the initiator being the compound with the formula tBuO—OtBu or tBuO—OC(O)tBu and the monomers being the compounds of formula I, II, III₁, III₂, II′, II″ and VI, the expression [initiator]ₒ expresses the initial molar concentration of initiator and the expression [monomers]ₒ expresses the total initial concentration of monomers.

13. Copolymerisation process according to any one of claims 6, 7, 8, 9, 11 and 12, characterised in that the reaction is conducted:
in the presence of t-butyl peroxypivalate and at a reaction temperature of between 70 and 80° C., preferably at a temperature of about 75° C.; or
in the presence of t-butyl peroxide and at a reaction temperature of between 135 and 145° C., preferably at a temperature of about 140° C.

14. Copolymerisation process according to any one of claims 6 to 9 and 11 to 13, characterised in that the reaction is carried out in the presence of at least one organic solvent.

15. Copolymerisation process according to claim 14, characterised in that the organic solvent is chosen from the group constituted by perfluoro-n-hexane, acetonitrile or mixtures of perfluoro-n-hexane and acetonitrile.

16. Copolymerisation process according to claim 14 or 15, characterised in that the content of solvent in the reaction medium is preferably such that the initial ratio by weight between the solvent and the monomers lies between 0.5 and 1.5, and preferably between 0.6 and 1.2.

17. Copolymerisation process according to any one of claims 6, 7, 8, 9, 11, 12 and 15, characterised in that the reaction is conducted with an initial molar ratio between the initiator and the. monomers ([initiator]ₒ/[monomers]ₒ) that lies between 0.1 and 2%, and preferably between 0.5 and 1%; the initiator being the compound with the formula tBuO—OtBu or tBuO—OC(O)tBu and the monomers being the compounds of formula I, II, III₁, III₂, II′, II″ and VI, the expression [initiator]ₒ expressing the initial molar concentration of initiator and the expression [monomers]ₒ expressing the total initial concentration of monomers.

18. Copolymerisation process according to claims 6, 7, 8, 9, 11, 12. 15 and 16, characterised in that the reagent with formula III₂ is perfluoro(4-methyl-3,6-dioxaoct-7-ene) sulphonyl fluoride (PFSO₂F) and that the compound of formula VI is vinylidene fluoride.

19. Fluorinated polymer, preferably fluorinated copolymer, capable of being obtained according to any one of claims 3 to 5.

20. Bromofunctional fluorinated copolymer capable of being obtained according to any one of claims 3 to 18.

21. Bromofunctional fluorinated copolymer according to claim 20, containing:
from 7 to 24% of bromotrifluoroethylene;
from 20 to 30% of perfluoro(4-methyl-3,6-dioxaoct-7-ene) sulphonyl fluoride, and
from 56 to 73% of vinylidene fluoride.

22. Bromofunctional fluorinated copolymer according to claim 20, containing:
from 2 to 15% of 1,1,2-trifluoro-4-bromobutene;
from 20 to 30% of perfluoro(4-methyl-3,6-dioxaoct-7-ene) sulphonyl fluoride; and
from 65 to 78% of vinylidene fluoride.

23. Bromofunctional fluorinated copolymers according to claim 20, characterised in that they possess the following chemical functions or fluorinated groups:
—SO₂F;
—OCF₂CF(CF₃)OCF₂CF₂SO₂F;
tBuO—CF2—CH₂—;
—CH₂CF₂—CH₂CF₂—CH₂—CF₂—;
—CH₂CF₂—CH₂CF₂—CF₂—CH₂—;
tBuO—CH₂CF₂—CH₂CF₂—;
—CH₂CF₂—(CF—CFBr)ₙ—;
—CF₂CF(OR_{F}SO₂F)—CH₂CF₂—CF₂CF(OR_{F}SO₂F)—;
—CH₂CF₂—CF₂CF(OR_{F}SO₂F)—;
—OCF₂CF(CF₃)OCF₂CF₂SO₂F;
—CH₂CF₂—CH₂CF₂—CF₂CH₂—;
—CH₂CF₂—CF₂CH₂—CH₂CF₂—;
—CF₂CFBrCH₂CF₂;
—CH₂CF₂—CF₂CF(OR_{F}SO₂F)—CH₂CF₂—;
—CH₂CF₂—CF₂CF(OR_{F}SO₂F)—CH₂CF₂—;
—(CF₂CFBr)ₙ—;
—CH₂CF₂—CF₂CF(OR_{F}SO₂F)—CF₂CH₂—; and
—OCF₂CF(CF₃)OC₂F₄SO₂F;
associated respectively with the following chemical shifts, expressed in ppm, in RMN of ¹⁹F:
+45;
−77 to −80;
−83;
−91;
−95;
−102;
−103 to 105;
−108;
−110;
−112;
−113;
−116;
−118;
−122;
−125;
−126;
−127; and
−144.

24. Bromofunctional fluorinated copolymers according to claim 20, characterised in that they possess the following chemical functions or fluorinated groups:
—SO₂F;
—OCF₂CF(CF₃)OCF₂CF₂SO₂F;
tBuO—CF2—CH₂—;
—CH₂CF₂—CH₂CF₂—CH₂CF₂—;
—CF₂CF(R_{F})—CH₂CF₂—CH₂CF₂—;
—CF₂CF (R_{F})—CH₂ CF₂(CH₂CF₂—CF₂CF₂—;
—CH₂CF₂—CH₂CF₂—CF₂CH₂—;
—CF₂CF(OR_{F}SO₂F)—CH₂CF₂—CF₂CF(OR_{F}SO₂F)—;
—CH₂CF₂—CH₂CF₂—CF₂CF(R₄)—;
—OCF₂CF(CF₃)OCF₂CF₂SO₂F;
—CH₂CF₂—CH₂CF₂—CF₂CH₂—;
—CH₂CF₂—CF₂CH₂—CH₂CF₂—;
—CH₂ CF₂—CF₂CF(C₂H₄Br)—CH₂CF₂—;
—CF₂CF(OR_{F}—SO₂F)—CF₂CF(C₂H₄Br)—CH₂—CF₂—;
—CH₂CF₂—CF₂CF(OR_{F}SO₂F)—CH₂CF₂—;
—CH₂CF₂—CF₂CF(OR_{F}SO₂F)—CH₂CF₂—;
—CH₂CF₂—CF₂CF(OR_{F}SO₂F)—CF₂CH₂—;
—OCF₂CF(CF₃)OC₂F₄SO₂F;
—CH₂CF₂—CF₂CF(C₂H₄Br)—CH₂CF₂—;
—CH₂CF₂—CF₂CF(C₂H₄Br)—CF₂—;
associated respectively with the following chemical shifts, expressed in ppm, in NMR of ¹⁹F:
+45;
−77 to −80;
−83;
−91;
−92;
−93;
−95;
−108;
−110;
−112;
−113;
−116;
−119;
−120;
−122;
−125;
−127;
−144;
−161 to −165 and
−178 to −182.

25. Process for preparing a bromosulphonated fluorinated elastomer, characterised in that the polymers obtained in any one of claims 3 to 18 is subjected to a cross-linking stage carried out preferably in the presence of at least one peroxide (preferably in a concentration of between 1 and 5%) and/or in the presence of at least one triallylisocyanurate (preferably in a concentration of between 5 and 20%) followed by a hot post-cross-linking stage preferably carried out at a temperature of between 200 and 220° C., limits included.

26. Bromosulphonated fluorinated elastomer capable of being obtained by the process of claim 25.

27. Bromosulphonated fluorinated elastomer, according to claim 25, characterised in that it exhibits very low glass transition temperatures (T_{g}), these glass transition temperatures, which are measured according to the standard ASTM E-1356-98, preferably lie between −45 and −18° C., more preferably still between −35 and −21° C.

28. Bromosulphonated fluorinated elastomer according to claim 25 or 26, characterised in that it exhibits an inherent viscosity measured according to the ASTM D-2857-95 method that lies between 0.8 and 1.8 mL/g.

29. Bromosulphonated fluorinated elastomer according to any one of claims 25 to 28, characterised in that it exhibits a thermal stability ATG of up to 325° C. in air at 10° C./min, at which temperature value weight loss of 5% is measured.

30. Use of one or more cross-linkable bromosulphonated fluorinated elastomers according to any one of claims 23 to 28, for:
the manufacture of membranes, polymeric electrolytes, ionomers, components of fuel cells supplied for example with hydrogen or methanol;
the making of sealing joints and O-rings, radiator hoses, pipes, pump housings, diaphragms, piston heads (for applications in the aeronautical, petroleum, automotive, mining, nuclear industries); and
for plastics processing (aid processing products).

31. Process for cross-linking the sulphonyl groups of a sulphonated polymer chosen from the family of the bromosulphonated fluorinated elastomers defined in any one of claims 25 to 29, during which process at least one fraction of the cross-linking bonds carries an ionic charge, said process including the bringing of said polymer into contact with a cross-linking agent permitting the reaction between two sulphonyl groups originating from adjacent polymer chains, in order to form said cross-linking bonds.
